(19) United States
(12) Patent Application Publication
(10) Pub. No.: US 2007/0111965 A1
(43) Pub. Date: May 17, 2007

(54) COMPOSITIONS COMPRISING LIPOXYGENASE INHIBITORS AND CYCLODEXTRIN

(76) Inventors: James E. Kipp, Wauconda, IL (US); Pramod Gupta, Pittsford, NY (US)

Correspondence Address: BAXTER HEALTHCARE CORPORATION, ONE BAXTER PARKWAY, DF2-2E, DEERFIELD, IL, 60015, US

(21) Appl. No.: 11/560,242

(22) Filed: Nov. 15, 2006

Related U.S. Application Data
(60) Provisional application No. 60/736,980, filed on Nov. 15, 2005.

(51) Int. Cl. A61K 31/724; A61K 31/381; A61K 31/343

(52) U.S. Cl. 514/58; 514/443; 514/469

(57) ABSTRACT

The present invention is directed to formulations of inclusion complexes of lipoxygenase inhibitors and cyclodextrins having a therapeutically effective concentration of the lipoxygenase inhibitor, methods of making the same and methods of treating disease states using the same. Forming cyclodextrin complexes permits the enhancement of the aqueous solubility of lipoxygenase inhibitors which allows higher concentrations of the lipoxygenase in solution. Aqueous formulations of lipoxygenase inhibitors-cyclodextrin complexes are suitable for parenteral or oral administration for treating and/or preventing inflammatory disease states. The aqueous formulations can be lyophilized to prolong storage stability, assist in oral administration and/or provide for convenient and economical packaging.

This application claims the benefit of U.S. Provisional Application Ser. No. 60/736,980 filed on Nov. 15, 2005.

### BACKGROUND OF THE INVENTION

The invention is directed to a composition comprising a lipoxygenase inhibitor and a cyclodextrin, an inclusion complex of cyclodextrin and a lipoxygenase inhibitor having a therapeutically effective concentration of the lipoxygenase inhibitor, pharmaceutical compositions thereof, methods of making a formulation of an inclusion complex of cyclodextrin and a lipoxygenase inhibitor having a therapeutically effective concentration of the lipoxygenase inhibitor, and therapeutic treatment methods using formulations of an inclusion complex of cyclodextrin and a lipoxygenase inhibitor having a therapeutically effective concentration of the lipoxygenase inhibitor. In particular, the invention is directed to formulations of an inclusion complex of a β-cyclodextrin or derivative thereof and a 5-lipoxygenase inhibitor having a therapeutically effective concentration of the lipoxygenase inhibitor, formulations of an inclusion complex of a β-cyclodextrin or derivative thereof and a 5-lipoxygenase inhibitor having a therapeutically effective concentration of the lipoxygenase inhibitor, methods of making formulations of an inclusion complex of a β-cyclodextrin or derivative thereof and a 5-lipoxygenase inhibitor having a therapeutically effective concentration of the lipoxygenase inhibitor and therapeutic treatment methods using formulations of an inclusion complex of a 13-cyclodextrin or derivative thereof and a 5-lipoxygenase inhibitor having a therapeutically effective concentration of the lipoxygenase inhibitor. These formulations can be made as aqueous solutions for administration via parenteral or oral routes, for example, or can be in dried form. The dried formulation can be reconstituted for administration or can be further processed for routes of administration including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal.

Lipoxygenase enzymes play an important role in various diseases such as asthma, rheumatoid arthritis, gout, psoriases, allergic rhinitis, Crohn's disease, respiratory distress syndrome, chronic obstructive pulmonary disease, acne, atherosclerosis, aortic aneurysm, sickle cell disease, acute lung injury, ischemia/reperfusion injury, nasal polyposis and/or inflammatory bowel disease among others. Accordingly, compounds which inhibit lipoxygenase activity are useful in the treatment and/or prevention of such diseases. U.S. Pat. Nos. 4,873,259, 4,992,464, and 5,250,565 which are incorporated herein by reference and made a part thereof, disclose certain lipoxygenase inhibitors, particularly 5- and/or 12-lipoxygenase inhibiting compounds, N-hydroxyurea 5- and/or 12-lipoxygenase inhibiting compounds, methods of making 5- and/or 12-lipoxygenase inhibiting compounds and pharmaceutical formulations of 5 and 12-lipoxygenase inhibitors. One such N-hydroxyurea lipoxygenase inhibitor is commonly known as zileuton. A solid dosage form of 600 mg zileuton for oral administration is used as a treatment for asthma.

Zileuton has the following chemical structure:

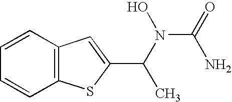

Zileuton may be used as a racemic mixture (about 50:50) of R(+) and S(−) enantiomers. Isomers of zileuton and their use in the inhibition of lipoxygenase activity have also been described. U.S. Pat. No. 5,629,337, which is incorporated herein by reference and made a part hereof, discloses the use of optically pure (-)-zileuton in the inhibition of lipoxygenase activity. WO 94/26268, which is incorporated herein by reference and made a part hereof, discloses the use of optically pure (+)-zileuton in the inhibition of lipoxygenase activity.

The low solubility in water of certain N-hydroxyurea 5- and/or 12-lipoxygenase inhibitors prevents these beneficial agents from broader use than they would otherwise enjoy if aqueous formulations could be prepared at therapeutically effective concentrations. Zileuton, for example, is soluble in methanol and ethanol, slightly soluble in acetonitrile, and practically insoluble in hexane and water (water solubility 0.08-0.14 mg/ml at 25° C.). In addition to its poor solubility, zileuton and likely other N-hydroxyurea lipoxygenase inhibitors are predicted to be chemically unstable in aqueous solution for storage at room temperature for prolonged periods of time [Insert Reference]. Degradation is consistent with specific hydronium- or water-catalyzed hydrolysis to afford the carbamic acid, which immediately loses carbon dioxide to generate the hydroxylamine as shown below.

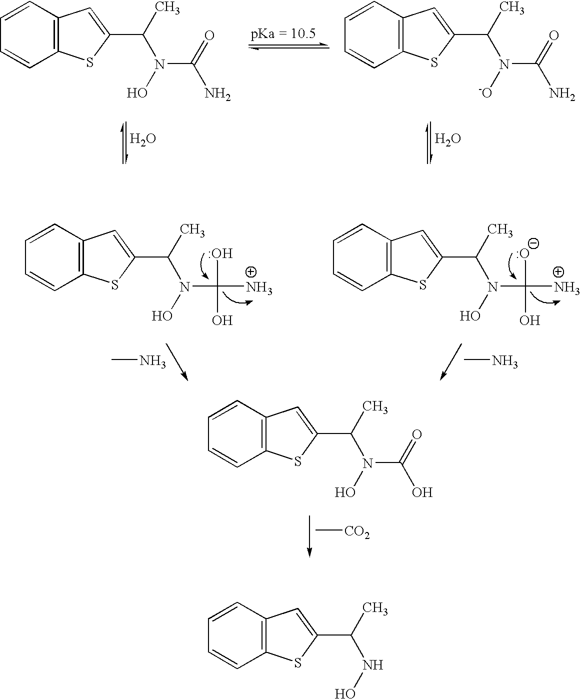

No buffer catalysis has been observed for zileuton. Using acid- and water-catalyzed rate constants at 25° C., the pseudo first-order rate constant is determined to be approximately 7.8×10⁻⁵ h⁻¹ over a pH range of about 3.5 to about 7.5. The shelf life at 10% drug loss is calculated to be 57.3 days at an optimal pH of 5.6. The pH-rate profile at 25° C., determined from rate data is as shown in FIG. 1.

Increasing the solubility of 5- and/or 12-lipoxygenase inhibitors such as zileuton can lead to increased therapeutic efficacy and increased therapeutic applications of the drug. For example, aqueous solutions having therapeutically effective concentrations of lipoxygenase inhibitors could be formulated into a ready-to-use injectable, such as an I.V. push or bolus injection. In addition, solution compositions could be prepared having higher concentrations of the lipoxygenase inhibitor for later dilution prior to injection. Injectable formulations of lipoxygenase inhibitors could permit its use in treating a broad array of disease states.

Once solution compositions having therapeutically effective concentrations of lipoxygenase inhibitors have been prepared, solid concentrates can be prepared by known methods. These soluble solid concentrates could then be dissolved at the time of injection. Also, these solid concentrates could be compounded to produce a single dosage form such as tablets, capsules, lozenges, suppositories, etc.

Therefore, there is a need for soluble or solution compositions of 5- and/or 12-lipoxygenase inhibitors having a therapeutically effective concentration of the lipoxygenase inhibitor for safe parenteral and/or oral administration, and in particular a soluble or solution composition having therapeutically effective concentrations of a 5-lipoxygenase inhibitor for parenteral administration. Moreover, a need exists for soluble or solution compositions of 5- and/or 12-lipoxygenase inhibitors which can provide therapeutically effective concentrations for parenteral administration without causing adverse effects from undesirably high concentrations of excipients.

### SUMMARY OF THE INVENTION

The present invention is directed to compositions comprising an inclusion complex of a lipoxygenase inhibitor and a cyclodextrin.

In another embodiment of the present invention, a pharmaceutical composition comprising an inclusion complex of a lipoxygenase inhibitor and cyclodextrin is provided, wherein the lipoxygenase inhibitor is present at a therapeutically effective concentration.

In another embodiment of the present invention, a pharmaceutical composition comprising an inclusion complex of a lipoxygenase inhibitor and a cyclodextrin is provided, wherein the lipoxygenase inhibitor is present at a therapeutically effective concentration and the cyclodextrin is selected from the group consisting of β-cyclodextrin, γ-cyclodextrin, γ-cyclodextrin and derivatives thereof.

In a further embodiment of the present invention, a pharmaceutical composition comprising an inclusion complex of a lipoxygenase inhibitor and a β-cyclodextrin or derivative thereof and a pharmaceutically acceptable excipient is provided, wherein the lipoxygenase inhibitor is present at a therapeutically effective concentration.

In another embodiment of the present invention, a pharmaceutical composition comprising an inclusion complex of a lipoxygenase inhibitor and a cyclodextrin and pharmaceutically acceptable excipient is provided, wherein the lipoxygenase inhibitor is present at a therapeutically effective concentration.

In yet another embodiment, a pharmaceutical composition comprising an inclusion complex of zileuton and a B-cyclodextrin and a pharmaceutically acceptable excipient is provided, wherein zileuton is present at a therapeutically effective concentration.

In another embodiment of the present invention, a parenteral formulation comprising an inclusion complex of a lipoxygenase inhibitor and a cyclodextrin is provided wherein the lipoxygenase inhibitor is present at a therapeutically effective concentration.

In yet another embodiment of the present invention, a dried formulation is provided comprising an inclusion complex of a lipoxygenase inhibitor and a cyclodextrin wherein the inclusion complex has a solubility of at least 0.2 mg/mL and the lipoxygenase inhibitor is present at a therapeutically effective concentration.

In yet another embodiment of the present invention, a method of making an aqueous solution of an inclusion complex of a 5-lipoxygenase inhibitor and a β-cyclodextrin comprising the steps of: preparing an aqueous buffer solution; dissolving a β-cyclodextrin derivative in the buffer solution; and adding a 5-lipoxygenase inhibitor to the β-cyclodextrin derivative and buffer solution is provided.

In another aspect of the present invention, a method of treating a mammal suffering from a condition mediated by lipoxygenase and/or leukotriene activity by administering the pharmaceutical composition comprising a lipoxygenase inhibitor and a cyclodextrin is provided, wherein said lipoxygenase inhibitor is present at a therapeutically effective concentration of the lipoxygenase inhibitor.

### BRIEF DESCRIPTION OF THE DRAWINGS

FIG. 1 shows the degradation reaction of zileuton in an aqueous solution.

FIG. 2 shows the pH-rate profile of zileuton at 25° C.

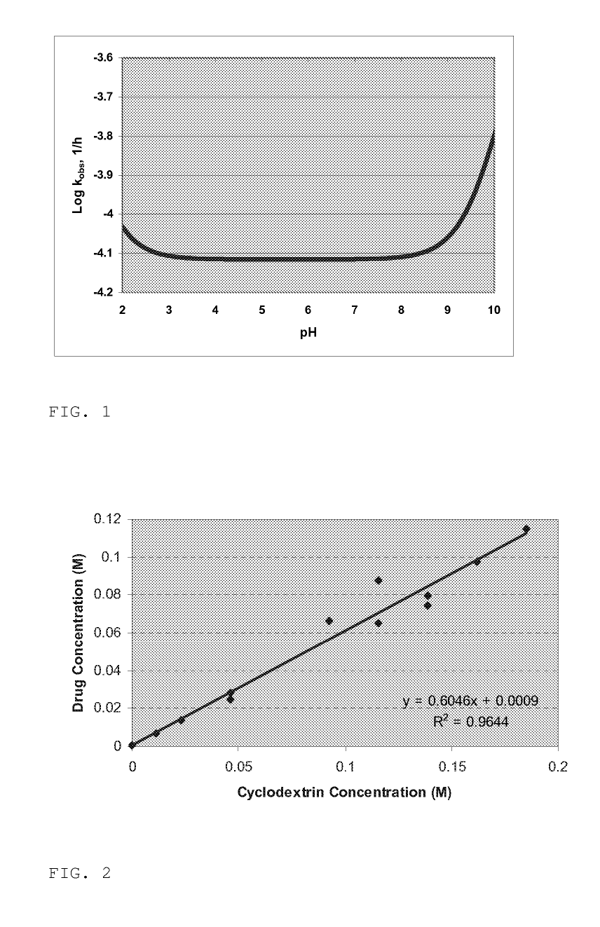

### DESCRIPTION OF THE INVENTION

As used herein, “a” or “an” are taken to mean one or more unless otherwise specified.

It has been determined that the desired solubility enhancement of 5- and/or 12-lipoxygenase inhibitors can be achieved by forming an inclusion complex with a cyclodextrin. Cyclodextrins were fully described by F. Schardinger and much of the older literature refers to cyclodextrins as Schardinger's dextrins. Cyclodextrins are cyclic oligosaccharides with hydroxyl groups on the outer surface and a cavity in the center. This cyclic orientation provides a truncated cone structure that is hydrophilic on the exterior and lipophilic on the interior.

The most common cyclodextrins are α-, β-, and γ-cyclodextrins, consisting of 6, 7 and 8 α-1,4-linked glucose units, respectively. The number of these units determines the size of the cavity.

Cyclodextrins are capable of forming inclusion complexes with hydrophobic molecules by taking up a whole molecule, or some part of it, into the cavity. The stability of the complex formed depends on how well the guest molecule fits into the cyclodextrin cavity. A composition comprising a lipoxygenase inhibitor and a cyclodextrin may include inclusion complexes of the lipoxygenase inhibitor and the cyclodextrin as well as lipoxygenase inhibitor and cyclodextrin that are not part of inclusion complexes.

α-, β-, and γ-cyclodextrins, have limited aqueous solubility and show some toxicity when given by injection. For example, although β-cyclodextrins form the most stable complex with many drugs, they have the lowest water solubility of the cyclodextrins. Therefore, to overcome these shortfalls, the cyclodextrin structure has been chemically modified to generate a safer cyclodextrin derivative with increased solubility. The modifications are typically made at one or more of the 2, 3, or 6 position hydroxyl groups. Cyclodextrin derivatives have, for example, been described in U.S. Pat. Nos. 5,134,127, 5,376,645, 5,571,534, 5,874,418, 6,046,177 and 6,133,248, the contents of which are herein incorporated by reference and made a part hereof. As used herein, the term “cyclodextrin” is intended to encompass unmodified cyclodextrins as well as chemically modified derivatives thereof.

Although α-, β- and γ-cyclodextrins can be used for complex formation with 5- and/or 12-lipoxygenase inhibitors, preferred cyclodextrins are the β- and γ-cyclodextrins and even more preferred are the β-cyclodextrins. Preferred β-cyclodextrins include 2-hydroxypropyl-β-cyclodextrin and sulfobutyl derivatized β-cyclodextrin (described, for example, in U.S. Pat. Nos. 5,134,127, 5,376,645, 5,874,418, 6,046,177 and 6,133,248). One such sulfobutyl derivatized β-cyclodextrin is sulfobutylether(7)-β-cyclodextrin. Sulfobutylether(7)-β-cyclodextrin is sold by CyDex, Inc. under the tradename CAPTISOL (“CAPTISOL Cyclodextrin”).

Preferred 5- and/ or 12-lipoxygenase inhibitors are of the type having the formula having the Formula (I):

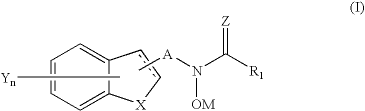

wherein R₁ is selected from the group consisting of hydrogen, C1-C4 alkyl, C2-C4 alkenyl, and NR₂R₃, wherein R₂ and R₃ are each independently selected from hydrogen, C1-C4 alkyl and hydroxyl, but R₂ and R₃ are not simultaneously hydroxyl;

wherein X is oxygen, sulfur, SO₂, or NR₄, wherein R₄ is selected from the group consisting of hydrogen, C1-C6 alkyl, C1-C6 alkoyl. aroyl and alkylsufonyl;

A is selected from C1-C6 alkylene and C2-C6 alkenylene;

n is 1-5;

each Y is independently selected from hydrogen, halo, hydroxyl, cyano, halosubstituted alkyl, C1-C12 alkyl, C2-C12 alkenyl, C1-C12 alkoxy, C3-C8 cycloalkyl, C1-C8 thioalkyl, aryl, aryloxy, aroyl, C1-C12 arylalkyl, C2-C12 arylalkenyl, C1-C12 arylalkoxy and C1-C12 arylthioalkoxy, wherein substitutents are selected from halo, nitro, cyano, C1-C12 alkyl, alkoxy and halosubstituted alkyl;

Z is oxygen or sulfur; and

M is hydrogen, a pharmaceutically acceptable cation, aroyl or C1-C12 alkoyl.

The substituent(s) Y and the linking group A may be attached at any available position of either ring.

In an additional embodiment, the 5- and/or 12-lipoxygenase inhibitors are of the type having the Formula (II): 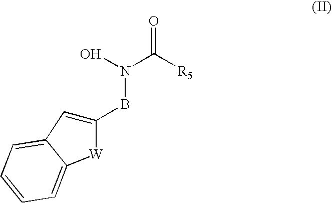
where R₅ is C1 or C2 alkyl, or NR₆R₇ where R₆ and R₇ are independently selected from hydrogen and C1 or C2 alkyl; B is CH₂ or CHCH₃ ; and W is oxygen or sulfur.

The term “alkylene” is used herein to mean straight or branched chain spacer radicals, for example, —CH₂—, —C(CH₃)₂—, —CH(C₂H₅)—, —CH₂CH₂—, —CH₂CHCH₃—, —C(CH₃)₂—,C(CH₃)₂—, CH₂CH₂CH₂.

The term “alkenylene” is used herein to mean straight or branched chain unsaturated spacer radicals, for example, —CH═CH—, —CH═CHCH₂—, CH═CHCH(CH₃)—, —C(CH₃)═CHCH₂—, —CH₂CH═CHCH₂—, —C(CH₃)₂CH═CHC(CH₃)₂—.

The term “alkyl” is used herein to mean straight or branched chain radicals of 1 to 12 carbon atoms, including, but not limited to methyl, ethyl, n-propyl, isopropyl, n-butyl, sec-butyl, isobutyl and tert-butyl.

The term “alkenyl” is used herein to mean straight or branched chain unsaturated radicals of 2 to 12 carbon atoms, including, but not limited to ethenyl, 1-propenyl, 2-propenyl, 2-methyl-1-propenyl, 1-butenyl, 2-butenyl.

The term “cycloalkyl” is used herein to mean cyclic radicals, for example, of 3 to 8 carbons, including, but not limited to cyclopropyl, cyclobutyl, cyclopentyl and cyclohexyl.

The term “alkoxy” is used herein to mean —OR₈ wherein R₈ is an alkyl radical, including, but not limited to methoxy, ethoxy, isopropoxy, n-butoxy, sec-butoxy, isobutoxy, tert-butoxy, and the like.

The term “thioalkyl” is used herein to mean —SR₉ wherein R₉ is an alkyl radical, including, but not limited to thiomethyl, thioethyl, thioisopropyl, n-thiobutyl, sec-thiobutyl, isothiobutyl and tert-thiobutyl.

The term “alkoyl” is used herein to mean —COR₁₀ wherein R₁₀ is an alkyl radical, including, but not limited to formyl, acetyl, propionyl, butyryl, isobutyryl and pivaloyl.

The term “carboalkoxy” is used herein to mean —COR₁₁, wherein R₁₁ is an alkoxy radical, including, but not limited to carbomethoxy, carboethoxy, carboisopropoxy, carbobutoxy, carbosec-butoxy, carboiso-butoxy and carbotert-butoxy.

The term “aryl” is used herein to mean substituted and unsubstituted carbocyclic and heterocylic aromatic radicals wherein the substituents are chosen from halo, nitro, cyano, alkyl, alkoxy, and halosubstituted alkyl, including, but not limited to phenyl, 1- or 2-naphthyl, 2-, 3-, or 4-pyridyl, 2- and 3-furyl.

The term “aroyl” is used herein to mean —COR₁₂ wherein R₁₂ is an aryl radical, including, but not limited to benzoyl, 1-naphthoyl and 2-naphthoyl.

The term “aryloxy” is used herein to mean —OR₁₃ wherein R₁₃ is an aryl radical, including, but not limited to phenoxy, 1-naphthoxy and 2-naphthoxy.

The term “arylalkoxy” is used herein to mean —OR₁₄ wherein R₁₄ is an arylalkyl radical, including, but not limited to phenylmethoxy (i.e., benzyloxy), 4-fluorobenzyloxy, 1-phenylethoxy, 2-phenylethoxy, diphenylmethoxy, 1-naphthylmethoxy, 2-napthylmethoxy, 9-fluorenoxy, 2-, 3- or 4-pyridylmethoxy and 2-, 3-, 4-, 5-, 6-, 7-, 8-quinolylmethoxy.

The term “arylthioalkoxy” is used herein to mean —SR₁₅ wherein R₁₅ is an arylalkyl radical, including, but not limited to phenylthiomethoxy (i.e., thiobenzyloxy), 4-fluorothiobenzyloxy, 1-phenylthioethoxy, 2-phenylthioethoxy, diphenylthiomethoxy and 1-naphthylthiomethoxy.

The term “arylalkyl” is used herein to mean an aryl group appended to an alkyl radical, including, but not limited to phenylmethyl (benzyl), 1-phenylethyl, 2-phenylethyl, 1-naphthylethyl and 2-pyridylmethyl.

The term “arylalkenyl” is used herein to mean an aryl group appended to an alkenyl radical, including, but not limited to phenylethenyl, 3-phenylprop-1-enyl, 3-phenylprop-2-enyl and 1-naphthylethenyl.

The term “alkylsulfonyl” is used herein to mean —SO₂ R₁₆ wherein R₁₆ is an alkyl radical, including, but not limited to methylsulfonyl (i.e. mesityl), ethyl sulfonyl and isopropylsulfonyl.

The terms “halo” and “halogen” are used herein to mean radicals derived from the elements fluorine, chlorine, bromine, or iodine.

The term “halosubstituted alkyl” refers to an alkyl radical as described above substituted with one or more halogens, including, but not limited to chloromethyl, trifluoromethyl, 2,2,2-trichloroethyl, and the like.

The term “pharmaceutically acceptable cation” refers to non-toxic cations including but not limited to cations based on the alkali and alkaline earth metals, such as sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine and ethylamine.

Inclusion complex formation of N-hydroxyurea 5- and/or 12-lipoxygenase inhibitors is favored since this class of lipoxygenase inhibitors has been shown to have therapeutic potential in clinical settings. Specifically, a preferred 5-lipoxygenase inhibitor, zileuton, has been clinical approved for the treatment of asthma by oral administration. Zileuton has the following chemical formula:

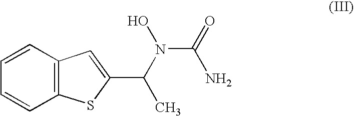

Certain of the lipoxygenase inhibitors described herein, including zileuton, contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. “Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. When the compounds described herein contain olefinic double bonds or other centers of geometric asymmetry, and unless specified otherwise, it is intended that the compounds include both E and Z geometric isomers. Likewise, all tautomeric forms are also intended to be included.

As used, herein, the term “zileuton” encompasses ((±)-1-(1-benzo[b]thien-2-ylethyl)-1-hydroxyurea, the optically pure form of the (S)-enantiomer or (-)-isomer of N-(1-benzo[b]thien-2-ylethyl)-N-hydroxyurea (described, for example, in U.S. Pat. No. 5,629,337), the optically pure form of (R)-enantiomer or (+)-isomer of N-(1-benzo[b]thien-2-ylethyl)-N-hydrxoyurea (described, for example, in WO 94/26268), mixtures of said (S)- and (R)-isomers in any ratio between 1:99 and 99:1, and polymorphic forms of zileuton that are now known or later discovered.

In one embodiment, the lipoxygenase inhibitor compound is selected from the group consisting of ((±)-1-(1-benzo[b]thien-2-ylethyl)-l-hydroxyurea, the optically pure (−)-isomer of N-(1-benzo[b]thien-2-ylethyl)-N-hydroxyurea and the optically pure (+)-isomer of N-(1-benzo[b]thien-2-ylethyl)-N-hydroxyurea.

In another embodiment of the present invention, a pharmaceutical composition comprising an inclusion complex of a lipoxygenase inhibitor and a cyclodextrin is provided having a therapeutically effective concentration of the lipoxygenase inhibitor. A therapeutically effective concentration as used herein means a concentration that provides a dosage of the drug that causes an ameliorative effect when administered to a subject for treatment or prevention of an inflammatory disease state without having to administer more than the typical maximum volume for the particular route of administration. With I.V. push formulations, for example, the concentration of the lipoxygenase inhibitor would have to be high enough to provide a dosage that causes an ameliorative effect without having to administer more than the typical maximum volume for an I.V. push of about 100 mL. The dosage is in turn dependent on a number of factors clinician take into consideration such as age, weight, diagnosis, disease stage, etc.

In one embodiment, a pharmaceutical composition comprising an inclusion complex of a lipoxygenase inhibitor and a cyclodextrin is provided, wherein the cyclodextrin is selected from the group consisting of α-cyclodextrins, β-cyclodextrins, γ-cyclodextrins and derivatives thereof. The inclusion complex is preferably formed of a 5-lipoxygenase inhibitor and a β-cyclodextrin or derivative thereof. In another embodiment, the pharmaceutical composition comprises a lipoxygenase inhibitor of Formula (I) and a β-cyclodextrin or derivative thereof, wherein the lipoxygenase inhibitor is present in a therapeutically effective amount. In another embodiment, the pharmaceutical composition comprises a lipoxygenase inhibitor of Formula (II) and a β-cyclodextrin or derivative thereof, wherein the lipoxygenase inhibitor is present in a therapeutically effective amount. Although many types of β-cyclodextrins can be used to form the complex, preferred β-cyclodextrins are hydroxypropyl-β-cyclodextrins and sulfobutyl derivatized β-cyclodextrins. A preferred lipoxygenase inhibitor and cyclodextrin inclusion complex is that of zileuton and sulfobutylether(7)-β-cyclodextrin.

The pharmaceutical compositions described herein can optionally include one or more pharmaceutically acceptable excipients. Such pharmaceutically acceptable excipients are well known in the art and include, for example, salts, surfactant(s), water-soluble polymers, preservatives, antimicrobials, antioxidants, cryo-protectants, wetting agents, viscosity agents, tonicity modifying agents, levigating agents, absorption enhancers, penetration enhancers, pH modifying agents, muco-adhesive agents, coloring agents, flavoring agents, diluting agents, emulsifying agents, suspending agents, solvents, co-solvents, buffers, and combinations of these excipients.

Suitable surfactants can be selected from ionic surfactants, nonionic surfactants, zwitterionic surfactants, polymeric surfactants, phospholipids, biologically derived surfactants, amino acids and their derivatives or derivatives, combinations or conjugates of the surfactants described above. Ionic surfactants can be anionic or cationic. The surfactants are present in the compositions in an amount of from about 0.01% to 10% w/v, and preferably from about 0.05% to about 5% w/v.

Suitable anionic surfactants include but are not limited to: alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidic acid and their salts, sodium carboxymethylcellulose, bile acids and their salts, cholic acid, deoxycholic acid, glycocholic acid, taurocholic acid, and glycodeoxycholic acid, and calcium carboxymethylcellulose, stearic acid and its salts, calcium stearate, phosphates, sodium dodecylsulfate, carboxymethylcellulose calcium, carboxymethylcellulose sodium, dioctylsulfosuccinate, dialkylesters of sodium sulfosuccinic acid, sodium lauryl sulfate and phospholipids.

Suitable cationic surfactants include but are not limited to: quaternary ammonium compounds, benzalkonium chloride, cetyltrimethylammonium bromide, chitosans, lauryldimethylbenzylammonium chloride, acyl camitine hydrochlorides, alkyl pyridinium halides, cetyl pyridinium chloride, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15-dimethyl hydroxyethyl ammonium chloride, C12-15-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12 trimethyl ammonium bromides, C15 trimethyl ammonium bromides, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, “POLYQUAT 10” (a mixture of polymeric quarternary ammonium compounds), , tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, “MIRAPOL” (polyquatemium-2) “Alkaquat” (alkyl dimethyl benzylammonium chloride, produced by Rhodia), alkyl pyridinium salts, amines, amine salts, imide azolinium salts, protonated quaternary acrylamides, methylated quaternary polymers, and cationic guar gum. benzalkonium chloride, dodecyl trimethyl ammonium bromide, triethanolamine, and poloxamines.

Suitable nonionic surfactants include but are not limited to: polyoxyethylene fatty alcohol ethers, polyoxyethylene sorbitan fatty acid esters, alkyl polyoxyethylene sulfates, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylmethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, p-isononylphenoxypoly(glycidol), decanoyl-N-methylglucamide, n-decyl-β-D-glucopyranoside, n-decyl -β-D-maltopyranoside, n-dodecyl -β-D-glucopyranoside, n-dodecyl-β-D-maltoside, heptanoyl-N-methylglucamide, n-heptyl-β-D-glucopy- ranoside, n-heptyl-β-D-thioglucoside, n-hexyl-β-D-glucopyranosid- e; nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof.

Suitable polymeric surfactants include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate), poly vinyl chloride polystyrene and polyvinylpryrrolidone.

Suitable biologically derived surfactants include, but are not limited to: lipoproteins, gelatin, casein, lysozyme, albumin, casein, heparin, hirudin, or other proteins.

Suitable buffers include, but are not limited to, sodium hydroxide, hydrochloric acid, tris buffer, mono-, di-, tricarboxylic acids and their salts, citrate buffer, phosphate buffer, glycerol-1-phosphate, glycercol-2-phosphate, acetate, lactate, tris(hydroxymethyl)aminomethane, aminosaccharides, mono-, di- and trialkylated amines, meglumine (N-methylglucosamine), and amino acids.

The pharmaceutical compositions described herein may be administered by several routes of administration including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal routes of administration. The route of administration as well as the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

The excipient included within the pharmaceutical compositions of the invention is chosen based on the expected route of administration of the composition in therapeutic applications. Accordingly, compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Solid dosage forms, such as tablets, pills and capsules, may also contain one or more binding agents, filling agents, suspending agents, disintegrating agents, lubricants, sweetening agents, flavoring agents, preservatives, buffers, wetting agents, disintegrants, effervescent agents, and other excipients. Such excipients are known in the art. Examples of filling agents are lactose monohydrate, lactose anhydrous, and various starches. Examples of binding agents are various celluloses and cross-linked polyvinylpyrrolidone, microcrystalline cellulose, microcrystalline cellulose, and silicifized microcrystalline cellulose (SMCC). Suitable lubricants, including agents that act on the flowability of the powder to be compressed, are colloidal silicon dioxide, talc, stearic acid, magnesium stearate, calcium stearate, and silica gel. Examples of sweeteners are any natural or artificial sweetener, such as sucrose, xylitol, sodium saccharin, cyclamate, aspartame, and accsulfame K. Examples of flavoring agents are bubble gum flavor, fruit flavors, and the like. Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride. Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, lactose such as lactose monohydrate, lactose anhydrous, dibasic calcium phosphate, mannitol, starch, sorbitol, sucrose and glucose. Suitable disintegrants include corn starch, potato starch, maize starch, and modified starches, croscarmellose sodium, crosspovidone, sodium starch glycolate, and mixtures thereof. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propyl parabens as preservatives, a dye and a flavoring such as cherry or orange flavor, and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administer to a wound site.

The compositions of the present invention can be administered parenterally such as, for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” is also meant to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

In one embodiment, the molar ratio of the lipoxygenase inhibitor to the cyclodextrin is preferably from about 10:1 to about 1:10. In another embodiment, the molar ratio of the lipoxygenase inhibitor is from about 5:1 to about 1:5. In yet another embodiment, the ratio is from about 1:1 to about 1:5. The concentration of the lipoxygenase inhibitor is preferably from about 0.1 mg/mL to about 200 mg/mL, more preferably from about 1 to about 100 mg/ml, more preferably from about 5 mg/mL to about 50 mg/mL and even more preferably from about 8 mg/mL to about 30 mg/mL and the concentration of the cyclodextrin is preferably from about 4 mM to about 900 mM, more preferably from about 20 mM to about 500 mM and even more preferably from about 30 mM to about 200 mM. In one embodiment, the lipoxygenase compositions of the present invention do not include a buffer. In another embodiment, the compositions optionally may include a buffer. Suitable buffer solutions include, but are not limited to, solutions of sodium hydroxide, hydrochloric acid, tris buffer, mono-, di-, tricarboxylic acids and their salts, citrate buffer, phosphate buffer, glycerol-1-phosphate, glycercol-2-phosphate, acetate, lactate, tris(hydroxymethyl)aminomethane, aminosaccharides, mono-, di- and trialkylated amines, meglumine (N-methylglucosamine), succinate, benzoate, tartrate, carbonate and amino acids. In a preferred embodiment, the buffer is a citrate buffer, and even more preferably a citrate buffer present at a concentration of from about 2 mM to about 500 mM. The compositions preferably have a pH of from about 3 to about 9. The compositions are preferably suited to be administered parenterally, and more preferably, administered as an I.V. push or bolus injection.

In another embodiment of the present invention, a method of making a pharmaceutical composition comprising an inclusion complex of a lipoxygenase inhibitor and a cyclodextrin is provided by preparing an aqueous buffer solution, dissolving a cyclodextrin in the buffer solution, and adding a lipoxygenase inhibitor to the cyclodextrin and buffer solution.

The method preferably further comprises stirring and/or sonicating the lipoxygenase inhibitor and cyclodextrin solution. The method also preferably comprises adjusting the pH of the buffer solution to be from about 3 to about 9. In one embodiment, the solution has a concentration of from about 0.1 mg/mL to about 200 mg/mL of the lipoxygenase inhibitor. In another embodiment, the concentration of lipoxygenase inhibitor is from about 5 mg/mL to about 50 mg/mL, and in yet another embodiment, the concentration is from about 8 mg/mL to about 30 mg/mL. In a further embodiment, the cyclodextrin is present at a concentration of from about 4 mM to about 900 mM, in another embodiment, from about 20 mM to about 500 mM and in yet another embodiment, from about 30 mM to about 500 mM. A preferred buffer is a citrate buffer present at a concentration of from about 2 mM to about 500 mM. In an additional embodiment, a composition comprising a lipoxygenase inhibitor and a cyclodextrin may comprise higher concentrations of a lipoxygenase inhibitor and a cyclodextrin than those described above. Such compositions can be diluted prior to administration to a patient.

A preferred lipoxygenase inhibitor is an N-hydroxyurea lipoxygenase inhibitor (described, for example, U.S. Pat. Nos. 4,873,259, 4,992,464, 5,250,565 and 5,629,337, and WO 94/26268). In a further embodiment, the lipoxygenase inhibitor is zileuton and the cyclodextrin is a β-cyclodextrin or derivative thereof. In a further preferred embodiment, the cyclodextrin is sulfobutylether(7)-β-cyclodextrin.

While it is possible to solubilize the lipoxygenase inhibitor in an excess of cyclodextrin when forming the inclusion complex, it can be desirable to minimize the amount of cyclodextrin needed to solubilize the drug, especially if the solution is to be administered parenterally.

In one embodiment, the stoichiometry of complexation of a drug-cyclodextrin complex is 1:1. In other words, the inclusion complex can include at least one molecule/mole of cyclodextrin for every molecule/mole of drug. In order to determine the minimum amount of cyclodextrin needed to solubilize the drug, a plot of drug solubility versus cyclodextrin concentration preferably should be carried out. From interpolation of the plot, a formulation can be prepared that minimally contains the amount of cyclodextrin needed to dissolve the lipoxygenase inhibitor. Since the stoichiometry of complexation will likely vary depending on the particular complex of 5- and/or 12-lipoxygenase inhibitor and cyclodextrin, it is desirable that such a solubility plot be conducted for each specific lipoxygenase-cyclodextrin complex. A solubility plot carried out on the zileuton-CAPTISOL Cyclodextrin complex is described below in Example 1.

Interpolation of the plot described in Example 1, the stoichiometry of complexation for the zileuton-CAPTISOL Cyclodextrin embodiment was determined to be about 1:1.8. In other words, the minimal amount of CAPTISOL Cyclodextrin needed to dissolve about one mole of zileuton in a preferred concentration range of about 5 to about 30 mg/mL is about 1.8 moles of CAPTISOL Cyclodextrin. As noted above, an excess of cyclodextrin can be used to dissolve the lipoxygenase inhibitor, particularly if the cyclodextrin does not produce any adverse effects upon administration of the formulation.

While a solution pH of 5.5 was initially selected for the zileuton-CAPTISOL Cyclodextrin complex, this may not be the case with other lipoxygenase-cyclodextrin complexes. As described in Example 2, further testing was done to determine an optimal pH range to maximize stability of the zileuton-CAPTISOL Cyclodextrin complex. Such testing may also be required to determine the optimal pH for other lipoxygenase-cyclodextrin complexes.

In addition to preparing solution formulations of lipoxygenase inhibitor-cyclodextrin complexes, solid formulations can be prepared by known methods, such as lyophilization, spray-drying and/or super-critical fluid extraction. These solid concentrates can then be re-suspended at the time of injection. Also, these solid concentrates may also be compounded to produce a single dosage form such as tablets, capsules, lozenges, suppositories, coated tablets, capsules, ampoules, suppositories, delayed release formulations, controlled release formulations, extended release formulations, pulsatile release formulations, immediate release formulations, gastroretentive formulations, effervescent tablets, fast melt tablets, oral liquid and sprinkle formulations. The solid concentrates may also be formulated in a form selected from the group consisting of a patch, a powder preparation for inhalation, a suspension, an ointment and an emulsion.

These dried formulations may be preferred for lipoxygenase inhibitor-cyclodextrin complexes that have poor long-term stability in solution form.

The dried formulation can be provided as is to the healthcare provider where it can be resolubilized in an appropriate diluent, such as a diluent suitable for parenteral or oral administration. The same formulation can be prepared by known methods for administration to a subject by various routes, such as, but are not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal.

In addition, the dried formulation can be resolubilized to produce a ready-to-use injectable formulation, preferably as an I.V. push or bolus injection. The lyophilized formulation can be resolubilized to a high concentration dosage which can be further diluted for injection. In a preferred embodiment, the lyophilized formulations are resolubilized for parenteral administration to provide a concentration range of the lipoxygenase inhibitor from about 0.1 to about 200 mg/mL, more preferably from about 5 to about 50 mg/mL, and even more preferably from about 8 to about 30 mg/mL

For the purpose of preparing a stabilized dry solid, bulking agents such as mannitol, sorbitol, sucrose, starch, lactose, trehalose or raffinose may be added prior to lyophilization. The solution may be lyophilized using any applicable program for lyophilization, for example:
loading at ±25° C.;
cooling down to −45° C. in 1 hour;
holding time at −45° C. for 3.5 hours;
mean drying for 33 hours with continual increase of temperature to +15° C. at a pressure of 0.4 mbar; and
final drying for 10 hours at +20° C. at a pressure of 0.03 mbar cryo protectant: mannitol.

Preferably, in order to aid in the selection of an appropriate lyophilization cycle for the particular lipoxygenase inhibitor-cyclodextrin complex solution freeze-thaw stability and DSC analysis of the solution formulation should be conducted.

Sterilization can be accomplished by a variety of methods known in the art including but not limited to heat sterilization, filtration, and irradiation. Sterilization can be accomplished by sterile filtration of the final lipoxygenase-cyclodextrin solution formulation. Any remaining steps, such as lyophilization or packaging, must then be carried out under sterile operating conditions. Typical sterile filtration methods include, for example, pre-filtration first through a 3.0 micrometer filter followed by filtration through a 0.45 micrometer particle filter, followed by filtration through two redundant 0.2 micrometer membrane filters.

The lipoxygenase inhibitor-cyclodextrin formulation whether as a solution formulation or a lyophilized formulation can be sterilized by heat sterilization, irradiation or other known sterilization methods, such as high pressure sterilization.

The pharmaceutical compositions described herein may be co-administered with one or more additional agents separately or in the same formulation. Such additional agents include, for example, anti-histamines, beta agonists (e.g., albuterol), antibiotics, anti-inflammatories (e.g. ibuprofen, prednisone (corticosteroid) or pentoxifylline), anti-fungals, (e.g. Amphotericin B, Fluconazole, Ketoconazol, and Itraconazol), steroids, decongestants, bronchodialators, and the like. The formulation may also contain preserving agents, solubilizing agents, chemical buffers, surfactants, emulsifiers, colorants, odorants and sweeteners.

The pharmaceutical composition described herein can be used to treat a patient suffering from a condition mediated by lipoxygenase and/or leukotriene activity. In one embodiment, the condition is mediated by 5- and/or 12-lipoxygenase activity. In another embodiment, the condition is an inflammatory condition.

Conditions mediated by lipoxygenase and/or leukotriene activity include, but are not limited to asthma, rheumatoid arthritis, gout, psoriases, allergic rhinitis, respiratory distress syndrome, chronic obstructive pulmonary disease, acne, atopic dermatitis, atherosclerosis, aortic aneurysm, sickle cell disease, acute lung injury, ischemia/reperfusion injury, nasal polyposis, inflammatory bowel disease (including, for example, ulcerative colitis and Crohn's disease), irritable bowel syndrome, cancer, tumors, respiratory syncytial virus, sepsis, endotoxin shock and myocardial infarction.

In one embodiment, the condition mediated by lipoxygenase and/or leuktoriene activity is an inflammatory condition. Inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, or Hodgkins disease.

In a further embodiment, the inflammatory condition is selected from the group consisting of rheumatoid arthritis, asthma, chronic obstructive pulmonary disease, acute lung injury, inflammatory bowel disease, allergy, organ ischemia, reperfusion injury, rhinitis, dermatitis, atherosclerosis, myocardial ischemia and adult respiratory distress syndrome.

### EXAMPLE 1

### Solubility Study

The solubility of zileuton at 5 and 25° C. in the presence of CAPTISOL Cyclodextrin was measured. A series of CAPTISOL Cyclodextrin solutions (100 to 400 mg/mL, or about 45 to 182 mM) were equilibrated with a molar excess of zileuton (100 mg/mL, or 423 mM). (See Table below.) Solutions were buffered, preferrably with 10 mM citrate buffer, to a pH of 5.5.

| Drug Concentration | CAPTISOL Cyclodextrin |
|---|---|
| (mg/mL) | concentration (mg/mL) |
| 100 | None |
| 100 | 25 |
| 100 | 50 |
| 100 | 100 |
| 100 | 250 |
| 100 | 300 |
| 100 | 350 |
| 100 | 400 |

These mixtures were sonicated and then stirred for 1 week at 5° C. Another similar set of samples, prepared as described above, were agitated in a controlled temperature chamber at 25° C.

After one week of equilibration, each sample was centrifuged, and the supernatant analyzed for drug concentration by simple UV assay. By plotting molar solubility of zileuton in each sample versus CAPTISOL Cyclodextrin concentration, the stoichiometry of complexation (1 :1 or 1:2, for example), and the binding constant, K was determined. For a 1:1 complex, the equation is [Higuchi T, Connors K A. Phase-solubility techniques. Adva Anal Chem Instr. 1965;4:212-217]:

$$S = {S}_{0} + \frac{K {S}_{0}}{1 + K {S}_{0}} {C}_{T}$$

S is the total drug solubility, bound to cyclodextrin and unbound, C_{T} is the total concentration of cyclodextrin in the sample, S₀ is the intrinsic solubility of the drug (solubility with cyclodextrin absent), and K is the 1:1 binding constant. From the slope, and knowledge of S₀, K can be determined. Results of this analysis are plotted in FIG. 2, and indicate a 1:1 binding constant of about 3,200 at 25° C. The molar ratio of cyclodextrin to drug at the solubility limit (25° C.) is approximately 1.7:1.

### EXAMPLE 2

### Stability and Stress Testing

A feasibility study to investigate the stability of zileuton-cyclodextrin solutions formulated at three different initial pH values (approximately 4.0, 5.5, and 7.0) was conducted. The solutions were formulated to contain 15 mg/mL zileuton, 250 mg/mL CAPTISOL Cyclodextrin, and 10 mM citrate buffer. Stress testing was performed by subjecting samples at each pH to both one and three freeze-thaw cycles. In addition, samples at each pH were stored at 5° C., 25° C., and 40° C. for a total of 8 weeks. At each testing interval, the samples were visually inspected and analyzed for pH, osmolality, color, and drug potency.

Zileuton-CAPTISOL Cyclodextrin formulations containing 15 mg/mL of drug and 250 mg/mL CAPTISOL Cyclodextrin were prepared at pH 4, 5.5, and 7, with an appropriate buffer, preferably 10 mM citrate, and stored at 5, 25 and 40° C for 8 weeks. Based on literature data [Alvarez, F J; Slade, R T. Kinetics and mechanism of degradation of zileuton, a potent 5-lipoxygenase inhibitor. Pharm. Res., 1992, 9(11): 1465-1473], zileuton in solution is expected to have adequate short-term stability (at least 1 month at 25° C.) over a pH range of 4 to 7.

A buffer stock solution (A) of 10 mM citric acid was prepared by adding distilled water to 1.9212 g citric acid anhydrous to a final volume of 1L. A buffer stock solution (B) of 10 mM sodium citrate was prepared by adding distilled water to 2.9411 g sodium citrate dihydrate (Na₃C₆H₅O₇.2H₂O) to a final volume of 1L.

The above stock buffer solutions A and B were combined to prepare buffer solutions for each formulation as shown in the Table 3 below:

TABLE 3
Preparation of Buffer Solutions

| | Citric Acid | Sodium Citrate | |
|---|---|---|---|
| Buffer | (mL) | (mL) | Measured pH |
| 10 mM citrate pH | q.s. to 200 mL | 72 | 3.94 |
| 4.0 ± 0.2 | | | |
| 10 mM citrate pH | 58 | q.s. to 200 mL | 5.47 |
| 5.5 ± 0.2 | | | |
| 10 mM citrate pH | 6 | q.s. to 200 mL | 6.97 |
| 7.0 ± 0.2 | | | |

The above buffer solutions were then used to make the three solutions at approximate pH 4.0, 5.5, and 7.0. All three solutions contained 15 mg/mL zileuton and 250 mg/mL CAPTISOL Cyclodextrin. Solution pH measurements were performed after addition and dissolution of zileuton and CAPTISOL Cyclodextrin (and sodium hydroxide for pH 5.5 and 7.0), but before final the final dilution step. Solutions were pipetted into amber glass vials and sealed with rubber stoppers and aluminum crimp caps. Vials were filled as 2-mL fill for potency testing and as 10-mL fill for measurement of pH, osmolality, color, and visual inspection. In addition, amber glass vials were filled (10 mL) for stress testing (freeze-thaw). All vials were stored in controlled temperature chambers at 5° C., 25° C., and 40° C.

Samples were pulled for testing at the time-zero, 1 week, 2 week, 4 week, and 8 week intervals.

Stress Testing (Freeze-Thaw)

Vials used for 1- and 3-cycle stress testing were stored at −20° C. for approximately 24 hours and then were placed in a 25° C. storage chamber for approximately 1 hr 20 minutes, at which point the samples were thawed. The 1-cycle stress samples were then tested for pH, osmolality, color, visual inspection and potency. The 3-cycle stress samples were placed back into the −20° C. chamber for approximately 24 hours and were then allowed to thaw at 25° C. for approximately 1 hour. The samples were placed back into the −20° C. chamber for approximately 26.5 hours and were then allowed to thaw at 5° C. for approximately 3 days until testing was performed for pH, osmolality, color, visual inspection and potency.

Results of the potency, pH, visual inspection, osmolality, and color testing for the 1-cycle and 3-cycle freeze-thaw stress testing are given in Tables 4-6.

TABLE 4
Freeze-Thaw Stress Data for pH 4.0 Solution

| Test | Potency | Measured | | Color | Osmolality |
|---|---|---|---|---|---|
| Interval | (mg/mL) | pH | Visual | (KSU) | (mOsmol/kg) |
| Time Zero | 15.14 | 4.06 | Pass | 0 | 864 |
| 1 Cycle | 14.58 | 4.07 | Pass | 20 | 862 |
| 3 Cycle | 15.21 | 4.05 | Pass | 16 | 868 |

TABLE 5
Freeze-Thaw Stress Data for pH 5.5 Solution

| Test | Potency | Measured | | Color | Osmolality |
|---|---|---|---|---|---|
| Interval | (mg/mL) | pH | Visual | (KSU) | (mOsmol/kg) |
| Time Zero | 14.64 | 5.61 | Pass | 19 | 852 |
| 1 Cycle | 14.46 | 5.54 | Pass | 19 | 857 |
| 3 Cycle | 14.60 | 5.47 | Pass | 19 | 864 |

TABLE 6
Freeze-Thaw Stress Data for pH 7.0 Solution

| Test | Potency | Measured | | Color | Osmolality |
|---|---|---|---|---|---|
| Interval | (mg/mL) | pH | Visual | (KSU) | (mOsmol/kg) |
| Time Zero | 14.51 | 6.93 | Pass | 15 | 863 |
| 1 Cycle | 14.41 | 6.94 | Pass | 15 | 860 |
| 3 Cycle | 14.55 | 6.97 | Pass | 17 | 866 |

The potency, pH, color, and osmolality data for the 1-cycle and 3-cycle freeze-thaw samples showed no significant changes. Furthermore, no significant particulates were observed upon visual inspection in any of the samples. Therefore, all samples appear to be stable against stresses imparted by freezing and thawing.

Stability of Samples Stored Through 8 weeks in Controlled Temperature Chambers

All 5° C. and 25° C. samples exhibited insignificant changes in potency over the 8 week storage period, and only modest pH changes, verifying the stability of these formulations at 5° C. and 25° C. across the entire storage period. Osmolality data indicated that the osmolality of these formulations ranged from 843-903 mOsmol/kg.

### EXAMPLE 3

The purpose of this study is to evaluate the stability of a zileuton-cyclodextrin solution, adjusted to an initial target pH of 4, and at lower drug and cyclodextrin levels (10 mg/mL zileuton, 167 mg/mL CAPTISOL Cyclodextrin), and buffered with 10 mM citrate.

A cyclodextrin solution was prepared by dissolving 417 g of CAPTISOL Cyclodextrin in approximately 1.75 L of 10 mM citrate buffer. 25 g of zileuton was weighed and transferred to the cyclodextrin solution with stirring. After complete dissolution, the formulation was tested for pH and confirmed to be at pH 4. The solution was then diluted with citrate buffer to bring the final volume of the solution to 2.5 L. An aliquot of this solution was tested for pH and was confirmed to be 4.

By a similar mixing procedure, a control solution was prepared without drug.

Glass vials were filled with the experimental and control formulations, and stored at 5° C., 25° C., and 40° C. Samples were pulled for testing at the time-zero, two-week, one-month, and three-month intervals. Testing was performed for potency, pH, visual inspection, osmolality (time-zero only), and color. Instrumental particle analysis was also performed at each interval.

The data indicated that the samples stored at 5 and 25° C. showed no significant change in drug level through 3 months. Visual inspection of the samples indicated no visible precipitation, or other phase separation. Instrumental particle counts demonstrated that the counts per mL for all solution units tested were within the current USP instrumental particle limits for 30 mL Small Volume Injection (SVI) solutions. The osmolality of the formulation at time-zero was 529 mOsmol/kg.

### EXAMPLE 4

### Stability of a zileuton-CAPTISOL Cyclodextrin Formulation Upon lyophilization, Followed by Reconstitution

The purpose of this study was to determine stability of a zileuton-cyclodextrin formulation (15 mg/mL zileuton, 250 mg/mL CAPTISOL Cyclodextrin, pH 4) that had been subjected to lyophilization. Lyophilized vials of zileuton-cyclodextrin formulation were reconstituted and analyzed to determine solution properties as a function of concentration. In addition, reconstituted vials were stored at two temperatures for two time points to investigate the stability of the reconstituted solutions. Samples were inspected visually and analyzed for pH, osmolality, color, and potency after reconstitution. Instrumental particle testing was performed immediately following reconstitution, as well as after storage at for 8 and 24 hours at 5 and 25° C., to look for evidence of precipitation. Testing was repeated after the lyophilized vials had been stored at 5° C. for approximately six months.

Lyophilized vial samples were reconstituted with diluent aliquots of 10, 15, and 20 mg/mL, and tested for potency, pH, osmolality, color, and visual inspection. Reconstituted vials were also tested for instrumental particle counts immediately after reconstitution, and after subsequent storage at 5 and 25° C., for 8 and 24 hours. Reconstitution was performed using filtered distilled water.

An additional test interval was conducted after the lyophilized vials had been stored for approximately 6 months at 5° C. Vials were reconstituted to 15 mg/mL with filtered distilled water and were tested for potency, pH, osmolality, color, and visual appearance. Vials were also examined for instrumental particle counts.

Results of potency, pH, visual inspection, osmolality, and color testing are given in Tables 7-10.

TABLE 7
Results for Samples Reconstituted with
15 mL Diluent (Initial Interval)

| | Potency | | | Color | Osmolality |
|---|---|---|---|---|---|
| Sample | (mg/mL) | PH | Visual | (ks) | (mOsmol/kg) |
| 15-A | 15.4 | 4.01 | pass | 10.0 | 960 |
| 15-B | 17.6 | 4.05 | pass | 7.0 | 967 |
| 15-C | 15.0 | 4.07 | pass | 10.0 | 976 |

TABLE 8
Results for Samples Reconstituted with
20 mL Diluent (Initial Interval)

| | Potency | | | Color | Osmolality |
|---|---|---|---|---|---|
| Sample | (mg/mL) | PH | Visual | (ks) | (mOsmol/kg) |
| 20-A | 11.5 | 3.98 | pass | 6.5 | 693 |
| 20-B | 11.7 | 3.98 | pass | 7.0 | 696 |
| 20-C | 11.4 | 3.97 | pass | 9.5 | 693 |

TABLE 9
Results for Samples Reconstituted with
30 mL Diluent (Initial Interval)

| | Potency | | | Color | Osmolality |
|---|---|---|---|---|---|
| Sample | (mg/mL) | pH | Visual | (ks) | (mOsmol/kg) |
| 30-A | 8.1 | 3.99 | pass | 4.0 | 447 |
| 30-B | 8.0 | 4.00 | pass | 3.5 | 447 |
| 30-C | 8.5 | 3.98 | pass | 5.0 | 447 |

TABLE 10
Results for Samples Reconstituted with
20 mL Diluent (6 Month Interval)

| | Potency | | | Color | Osmolality |
|---|---|---|---|---|---|
| Sample | (mg/mL) | pH | Visual | (ks) | (mOsmol/kg) |
| 20-A(6 MO) | 12.26 | 4.15 | pass | 17 | 687 |
| 20-B(6 MO) | 12.32 | 4.16 | pass | 15 | 688 |
| 20-C(6 MO) | 12.20 | 4.16 | pass | 17 | 687 |

Zileuton concentration in the reconstituted samples were consistent with the dilution factors, when accounting for the volume occupied by the lyophilizate (drug and CAPTISOL Cyclodextrin). The pH data for the reconstituted vials indicated that all solutions have a pH of 4.0±0.1, after reconstitution. Osmolality data shows an increase in osmolality with increasing formulation concentration (decreasing diluent volume).

Potency values for samples reconstituted after six months storage were consistent with stable product. The pH data at the six month interval indicated an insignificant change in pH. Osmolality data was consistent with the data from the initial interval.

All vials passed visual inspection. The instrumental particle counts per mL for all of the samples tested were within the current USP particle limits for 20 mL Small Volume Injection (SVI) solutions.

While the present invention has been described with references to certain preferred embodiments, these preferred embodiments are in no way meant to limit the scope of the present invention in any way. The scope of the present invention is defined by the claims which follow and all equivalents to which they are entitled under law.

1. A pharmaceutical composition comprising an inclusion complex of a lipoxygenase inhibitor and a cyclodextrin, wherein the lipoxygenase inhibitor is present in the composition at a therapeutically effective concentration.

2. The pharmaceutical composition of claim 1 further including a pharmaceutically acceptable excipient.

3. The pharmaceutical composition of claim 1 wherein the lipoxygenase inhibitor is selected from the group consisting of a 5-lipoxygenase inhibitor, a 12-lipoxygenase inhibitor and an inhibitor of 5- and 12-lipoxygenase.

4. The pharmaceutical composition of claim 3 wherein the cyclodextrin is selected from the group consisting of α-cyclodextrin, β-cyclodextrin, γ-cyclodextrin and derivatives thereof.

5. The pharmaceutical composition of claim 4 wherein the lipoxygenase inhibitor is a 5-lipoxygenase inhibitor.

6. The pharmaceutical composition of claim 5 wherein the cyclodextrin is a β-cyclodextrin or a derivative thereof.

7. The pharmaeutical composition of claim 3 wherein the lipoxygenase inhibitor has the Formula (II): 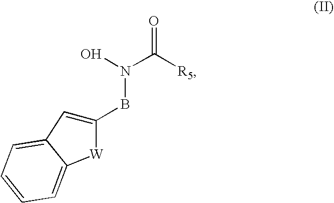
wherein R₅ is C₁ or C₂ alkyl or NR₆R₇, where R₆ and R₇ are independently selected from hydrogen and C₁ or C₂ alkyl; B is CH₂ or CHCH₃ ; and W is oxygen or sulfur.

8. The pharmaceutical composition of claim 7 wherein the cyclodextrin is selected from the group consisting of a 2-hydroxypropyl-β-cyclodextrin and a sulfobutyl derivatized β-cyclodextrin.

9. The pharmaceutical composition of claim 8 wherein the lipoxygenase inhibitor has the Formula (III):

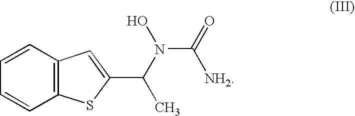

10. The pharmacuetical composition of claim 9 wherein the β-cyclodextrin is sulfobutylether(7)-β-cyclodextrin.

11. The pharmaceutical composition of claim 10 wherein the concentration of the lipoxygenase inhibitor is from about 0.1 mg/mL to about 200 mg/mL.

12. The pharmaceutical composition of claim 12 wherein the concentration of the lipoxygenase inhibitor is from about 5 mg/mL to about 50 mg/mL.

13. The pharmaceutical composition of claim 12 wherein the molar ratio of the lipoxygenase inhibitor to the cyclodextrin is from about 10:1 to about 1: 10.

14. The pharmaceutical composition of claim 13 wherein the 5-lipoxygenase inhibitor is present at a concentration of from about 0.1 mg/mL to about 200 mg/mL and the cyclodextrin is present at a concentration of from about 10 mg/mL to about 500 mg/mL.

15. The pharmaceutical composition of claim 14 further comprising a buffer.

16. The pharmaceutical composition of claim 15 wherein the buffer is a citrate buffer.

17. The pharmaceutical composition of claim 16 wherein the concentration of the citrate buffer is from about 5 mM to about 500 mM.

18. The pharmaceutical composition of claim 17 having a pH of from about 3 to about 9.

19. The pharmaceutical composition of claim 18 formulated for parenteral administration.

20. A parenteral formulation comprising an inclusion complex of a lipoxygenase inhibitor and a cyclodextrin wherein the lipoxygenase inhibitor is present at a therapeutically effective concentration.

21. The parenteral formulation of claim 20 wherein the lipoxygenase inhibitor is a 5-lipoxygenase inhibitor and the cyclodextrin is a 13-cyclodextrin or derivative thereof.

22. The parenteral formulation of claim 21 wherein the molar ratio of the 5-lipoxygenase inhibitor to the 13-cyclodextrin is from about 10:1 to about 1: 10.

23. The parenteral formulation of claim 22 wherein the concentration of the 5-lipoxygenase inhibitor is from about 0.1 mg/mL to about 200 mg/mL and the concentration of 13-cyclodextrin is from about 4 mM to about 900 mM.

24. The parenteral formulation of claim 23 wherein the concentration of the 5-lipoxygenase inhibitor is from about 5 mg/mL to about 50 mg/mL and the 13-cyclodextrin is present at a concentration of from about 20 mM to about 500 mM.

25. The parenteral formulation of claim 24 further comprising a buffer.

26. The parenteral formulation of claim 25 wherein the buffer is a citrate buffer present at a concentration of from about 5 mM to about 500 mM.

27. The parenteral formulation of claim 26 wherein the 5-lipoxygenase inhibitor is present at a concentration of from about 0.1 mg/mL to about 200 mg/mL, the β-cyclodextrin is present at a concentration of from about 10 mM to about 500 mM, the citrate buffer is present at a concentration of from about 5mM to about 15mM and wherein the parenteral formulation has a pH of from about 3 to about 9.

28. The parenteral formulation of claim 27 wherein the β-cyclodextrin is selected from the group consisting of a 2-hydroxypropyl-β-cyclodextrin and a sulfobutyl derivatized β-cyclodextrin and the 5-lipoxygenase inhibitor has the formula (III):

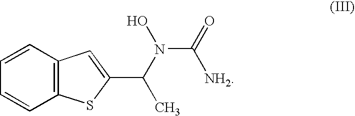

29. A dried formulation comprising an inclusion complex of a lipoxygenase inhibitor and a cyclodextrin wherein the inclusion complex has a solubility of at least 0.2 mg/mL and the lipoxygenase inhibitor is present at a therapeutically effective concentration.

30. The dried formulation of claim 29 wherein the lipoxygenase inhibitor is a 5-lipoxygenase inhibitor and the cyclodextrin is a β-cyclodextrin.

31. The dried formulation of claim 30 wherein the β-cyclodextrin is selected from the group consisting of 2-hydroxypropyl-β-cyclodextrins and sulfobutyl derivatized β-cyclodextrins and the 5-lipoxygenase inhibitor has the formula

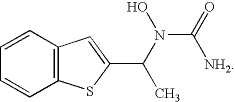

32. The dried formulation of claim 31 further comprising a buffer.

33. The dried formulation of claim 32 wherein upon dissolution with an aqueous diluent the concentration of the 5-lipoxygenase inhibitor is from about 0.1 mg/mL to about 200 mg/mL.

34. The dried formulation of claim 33 adapted for oral, rectal, nasal, pulmonary, ophthalmic, vaginal, aural, topical, buccal, transdermal, intravenous, intramuscular, subcutaneous, intradermal, intraocular, intracerebral, intralymphatic, intraarticular, intrathecal or intraperitoneal administration.

35. The dried formulation of claim 29 wherein said formulation is prepared by a method selected from the group consisting of lyophilization, spray-drying and super-critical fluid extraction,

36. A composition comprising an inclusion complex of a lipoxygenase inhibitor and a cyclodextrin.

37. The composition of claim 36 wherein the lipoxygenase inhibitor has the Formula (II):

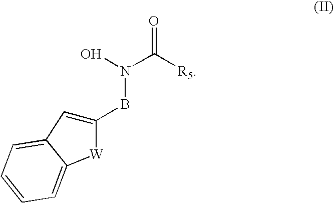

38. The composition of claim 37 wherein the cyclodextrin is selected from the group consisting of α-cyclodextrin, β-cyclodextrin, γ-cyclodextrin and derivatives thereof.

39. The composition of claim 38 wherein the cyclodextrin is a β-cyclodextrin or a derivative thereof.

40. The composition of claim 38 wherein the cyclodextrin is selected from the group consisting of a 2-hydroxypropyl-β-cyclodextrin and a sulfobutyl derivatized β-cyclodextrin.

41. A method of making an aqueous solution of an inclusion complex of a lipoxygenase inhibitor and a β-cyclodextrin comprising the steps of:
a. preparing an aqueous buffer solution;
b. dissolving a β-cyclodextrin in the buffer solution; and
c. adding a lipoxygenase inhibitor to the β-cyclodextrin and buffer solution to create a mixture thereof.

42. The method of making the aqueous solution of claim 41 further comprising the step of stirring and/or sonicating the mixture of lipoxygenase inhibitor and the β-cyclodextrin.

43. The method of making the aqueous solution of claim 41 further comprising the step of adjusting the pH of the buffer solution to be from about 3 to about 9.

44. The method of making an aqueous solution of claim 42 wherein the solution has a concentration of 0.1 mg/mL to about 200 mg/mL of the 5-lipoxygenase inhibitor, a concentration of from about 10 mM to about 500 mM of the β-cyclodextrin, and wherein the buffer is a citrate buffer present at a concentration of from about 5 mM to about 15 mM.

45. The method of making an aqueous solution of claim 43 wherein the β-cyclodextrin is selected from the group consisting of a 2-hydroxypropyl-β-cyclodextrin and a sulfobutyl derivatized β-cyclodextrin and the 5-lipoxygenase inhibitor has the formula (III):

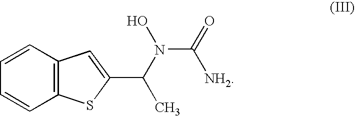

46. A method of treating a condition mediated by lipoxygenase activity in a mammal in need thereof comprising the steps of administering a formulation comprising an inclusion complex of a lipoxygenase inhibitor and cyclodextrin, wherein said formulation includes a therapeutically effective concentration of the lipoxygenase inhibitor.

47. The method of claim 46 wherein the condition is selected from the group consisting of asthma, rheumatoid arthritis, gout, psoriases, allergic rhinitis, respiratory distress syndrome, chronic obstructive pulmonary disease, acne, atopic dermatitis, atherosclerosis, aortic aneurysm, sickle cell disease, acute lung injury, ischemia/reperfusion injury, nasal polyposis, inflammatory bowel disease, irritable bowel syndrome, cancer, tumors, respiratory syncytial virus, sepsis, endotoxin shock and myocardial infarction.

48. The method of claim 46, wherein the condition is an inflammatory condition.

49. The method of claim 46 wherein the lipoxygenase inhibitor is selected from the group consisting of a 5-lipoxygenase inhibitor, a 12-lipoxygenase inhibitor and an inhibitor of 5- and 12-lipoxygenase and the cyclodextrin is selected from the group consisting of α-cyclodextrin, β-cyclodextrin and 7-cyclodextrin or a derivative thereof.

50. The method of claim 46 wherein the formulation is an aqueous solution and the lipoxygenase inhibitor is present at a concentration of about 0.1 mg/mL to about 200 mg/mL.

51. The method of claim 46 wherein the cyclodextrin is selected from the group consisting of a 2-hydroxypropyl-β-cyclodextrin and a sulfobutyl derivatized β-cyclodextrin and the lipoxygenase inhibitor has the formula (III):

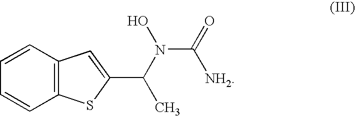

52. The method of claim 51 wherein the formulation is administered parenterally.

53. The method of claim 52 wherein the formulation is a lyophilizate.

54. The method of claim 53 wherein the formulation is administered orally.
